(19) United States
(12) Patent Application Publication
(10) Pub. No.: US 2024/0426834 A1
(43) Pub. Date: Dec. 26, 2024

(54) USE OF POLYPEPTIDES WITH CALCIUM INDICATOR ACTIVITY FOR IDENTIFYING THE ACTIVITY OF INSECTICIDAL PROTEINS

(71) Applicant: BAYER AKTIENGESELLSCHAFT, Leverkusen (DE)

(72) Inventors: Klaus Raming, Leverkusen (DE); Anke Koopmann, Leverkusen (DE); Ralf Nauen, Leverkusen (DE)

(21) Appl. No.: 18/683,918

(22) PCT Filed: Aug. 17, 2022
(86) PCT No.: PCT/EP2022/072992

§ 371 Date: Feb. 15, 2024

(30) Foreign Application Priority Data
Aug. 20, 2021 (EP) EP21192283.6

(51) Int. Cl. G01N 33/68; C07K 14/435; C07K 14/47

(52) U.S. Cl. CPC G01N 33/6845; C07K 14/43563; C07K 14/4728; G01N 2333/43552; G01N 2500/10

(57) ABSTRACT

Provided is the use of a polypeptide with calcium indicator activity which comprises an amino acid sequence represented by SEQ ID NO. 1, or an amino acid sequence which has at least 80% sequence identity thereto, for identifying transmembrane pore formation capability of a target polypeptide, especially an insecticidal protein, in a cellular assay.

The present application is a 35 U.S.C. § 371 national phase entry of International Application No. PCT/EP2022/072992, filed on Aug. 17, 2022, which claims priority to European Patent Application No. 21192283.6, filed Aug. 20, 2021, the contents of each of which are hereby incorporated by reference in their entirety.

### REFERENCE TO SEQUENCE LISTING SUBMITTED ELECTRONICALLY

A sequence listing contained in the file named “REPLACEMENT_09102024.xml”, created on Sep. 10, 2024, which is 22 kilobytes (measured in MS-Windows®) and comprises 11 sequences, is incorporated herein by reference in its entirety.

The present invention relates to the use of a polypeptide with calcium indicator activity which comprises an amino acid sequence represented by SEQ ID NO. 1, or an amino acid sequence which has at least 80% sequence identity thereto, for identifying transmembrane pore formation capability of a target polypeptide, especially an insecticidal protein (IP), in a cellular assay.

Insect-protected plants expressing insecticidal proteins derived from the entomopathogenic bacterium Bacillus thuringiensis (Bt) have transformed farming practices in many countries. The insecticidal traits resulting from transgene expression of insecticidal proteins provide these crops with robust and effective protection from insect herbivory. When ingested by a susceptible insect these proteins become activated by gut proteases, bind to cognate receptors in the insect gut, and form transmembrane pores that eventually kill the insect (Vachon et al., 2012; Pardo-Lopez et al., 2013). Activity assays and the knowledge of the exact Mode of action (MOA) is a key component in identifying new insecticidal proteins for deployment in next-generation insect-protected crops as well as for resistance research activities validating target site mutations in expressed receptor proteins.

There are several published in vitro methods to study IP activity and MOA including insect cell-based assays using native cells or heterologous expressed insect receptor genes (Tanaka et al., 2013; Onofre et al., 2017, Tanaka et al., 2016, ang et al.; 2019). Here, osmotic swelling assays, two-electrode voltage clamp technique in oocytes, cytotoxicity detection with fluorescent probes or binding assays have been used for insecticidal protein characterization. However, the methods mentioned are disadvantageous, for example with regard to simplicity, fastness, high-throughput applicability and/or invasiveness.

Genetically encoded calcium indicators are also known in the art. As a protein that functions as a calcium sensor, a calcium indicator protein is known in which a partial sequence of calmodulin and a partial sequence of myosin light chain kinase are linked to a fluorescent protein. This calcium indicator protein utilizes the phenomenon in which binding of calcium to the partial sequence of calmodulin causes a change in the conformation of the protein, which causes a change in the intensity of fluorescence emitted by the fluorescent protein (green fluorescent protein GFP or red fluorescent protein RFP). For example, Nakai J et al. developed a calcium indicator being a synthetic fusion protein consisting of three key domains: an M13 domain (which is a peptide sequence from myosin light-chain kinase) at the N-Terminus, a green fluorescent protein (GFP) in the center, and a calmodulin (CaM) domain at the C-Terminus (Nakai J et al., Nature Biotechnology. 19 (2): 137-41, 2001). Such calcium indicators are used to measure intracellular Ca²⁺ levels both in vitro and in vivo. The genetic sequence encoding such calcium indicator can be inserted under the control of promoters exclusive to certain cell types, allowing for cell-type specific expression. In the meantime, a variety of genetically encoded calcium indicators have been described. However, such molecules have not been used successfully to study insecticidal protein activity and MOA in insect cell-based assays, especially not in insect cell-based assays which are suitable for high throughput screening.

It was therefore an object of the present invention to provide polypeptides having calcium ion (Ca²⁺) indicator activity for use in cellular assays, especially in easy-to-use, fast, high-through-put applicable and/or non-invasive cellular assays.

Therefore, the invention is directed to the use of a polypeptide which
(i) comprises an amino acid sequence represented by SEQ ID NO. 1, or
(ii) comprises an amino acid sequence which has at least 80% sequence identity with the amino acid sequence of (i) over its entire length as determined using the BLASTX/ClustalW alignment tool, wherein said amino acid sequence has calcium ion (Ca²⁺) indicator activity,
   for identifying transmembrane pore formation capability of a target polypeptide in a cellular assay.

Using such polypeptide according to the invention allows for identifying transmembrane pore formation capability of a target polypeptide in a cellular assay which is easy-to-use, fast, high-through-put applicable and non-invasive. In a specific embodiment, said cellular assay is used to characterize insecticidal proteins.

By using such a polypeptide according to the invention, the activity of the target protein and the corresponding pore formation is measured by the calcium influx through the formed pores. Therefore, the polypeptide according to the invention has calcium ion (Ca²⁺) indicator activity. Preferably, it is a genetically encoded calcium ion (Ca²⁺) indicator.

Unexpectedly, the use of such genetically encoded calcium ion (Ca²⁺) indicators in an assay according to the invention turned out to be highly advantageous. In general, the variation of intracellular calcium can be measured in isolated cells by different approaches, including for example optical measurements of free cytosolic calcium fluctuations by using a collection of synthetic organic molecules that change fluorescence or absorbance properties upon calcium binding. However, such synthetic indicators are difficult to target to specific cell types or sub-cellular locations. Further, the loading procedures are invasive and damaging precluding repeated, chronic in vivo measurements. In contrast, using polypeptides according to the invention as described will overcome said disadvantages

The polypeptide to be used according to the invention comprises an amino acid sequence represented by SEQ ID NO. 1, or comprises an amino acid sequence which has at least 80% sequence identity with the amino acid sequence represented by SEQ ID NO. 1 over its entire length.

In another embodiment, the polypeptide to be used according to the invention comprises an amino acid sequence which has at least 81%, 82%, 83%, 84%, 85%, 86%, 87%, 88%, 89%, 90%, 91%, 92%, 93%, 94%, 95%, 96%, 97%, 98%, 99% or 99.5% sequence identity with the amino acid sequence represented by SEQ ID NO. 1 over its entire length. It is important that such amino acid sequence still has calcium ion (Ca²⁺) indicator activity.

SEQ ID NO. 1 refers to a calcium indicator (GCaMP) for imaging activity which has been optimized for neuronal populations and microcompartments as described (Dana et al., Nature Methods, 2019 July; 16(7):649-657).

Preferably, the polypeptide to be used according to the invention provides a fluorescent signal upon calcium ion (Ca²⁺) binding, i.e. the read-out for the calcium ion (Ca²⁺) indicator activity of said polypeptide is a fluorescent signal. Therefore, the polypeptide to be used according to the invention preferably comprises a fluorescent protein domain, for example green fluorescent protein, yellow fluorescent protein, etc.

In cells susceptible for insecticidal proteins, preferably insect cells, the formation of transmembrane pores by addition of insecticidal proteins gives rise to an intracellular increase of calcium under high extracellular calcium concentrations. Calcium ions flow across the membrane through the new build pores into the cell. The measurement of the increase of intracellular calcium in these cells correlates with the activity of the insecticidal protein. Cells not susceptible to insecticidal proteins will not react with such a calcium increase after addition of such insecticidal protein.

In one embodiment, the cells in the cellular assay are native cells, preferably native insect cells. Said cells are susceptible for the target polypeptide, i.e. preferably the insecticidal protein.

In another embodiment, the cells in the cellular assay are cells, preferably insect cells, which heterologously express at least one insect receptor gene. Because of the expression of said at least one insect receptor gene, said cells become susceptible for the target polypeptide, i.e. preferably the insecticidal protein, as well. The at least one insect receptor gene can preferably be a toxin receptor gene. Of course, it is possible that such cells heterologously express more than one insect receptor gene.

Heterologous expression refers to the expression of a gene or part of a gene in a host cell which does not naturally have this gene or gene fragment. Insertion of the gene in said heterologous host cells is performed by recombinant DNA technology. After being inserted in the host cell, the gene may be integrated into the host cell DNA, causing permanent expression, or not integrated, causing transient expression.

In a preferred embodiment, the cells in the cellular assay comprise an insect receptor polypeptide which
(i) comprises an amino acid sequence represented by SEQ ID NO. 2 or SEQ ID NO. 3 or SEQ ID NO. 10 or SEQ ID NO. 11, or
(ii) comprises an amino acid sequence which has at least 80% sequence identity with one of the amino acid sequences of (i) over its entire length as determined using the BLASTX/ClustalW alignment tool.

In another preferred embodiment, the insect receptor polypeptide comprises an amino acid sequence which has at least 81%, 82%, 83%, 84%, 85%, 86%, 87%, 88%, 89%, 90%, 91%, 92%, 93%, 94%, 95%, 96%, 97%, 98%, 99% or 99.5% sequence identity with the amino acid sequence represented by SEQ ID NO. 2 over its entire length.

In another preferred embodiment, the insect receptor polypeptide comprises an amino acid sequence which has at least 81%, 82%, 83%, 84%, 85%, 86%, 87%, 88%, 89%, 90%, 91%, 92%, 93%, 94%, 95%, 96%, 97%, 98%, 99% or 99.5% sequence identity with the amino acid sequence represented by SEQ ID NO. 3 over its entire length.

In another preferred embodiment, the insect receptor polypeptide comprises an amino acid sequence which has at least 81%, 82%, 83%, 84%, 85%, 86%, 87%, 88%, 89%, 90%, 91%, 92%, 93%, 94%, 95%, 96%, 97%, 98%, 99% or 99.5% sequence identity with the amino acid sequence represented by SEQ ID NO. 10 over its entire length.

In another preferred embodiment, the insect receptor polypeptide comprises an amino acid sequence which has at least 81%, 82%, 83%, 84%, 85%, 86%, 87%, 88%, 89%, 90%, 91%, 92%, 93%, 94%, 95%, 96%, 97%, 98%, 99% or 99.5% sequence identity with the amino acid sequence represented by SEQ ID NO. 11 over its entire length.

The target polypeptide according to the invention can be any polypeptide. Using a polypeptide comprising SEQ ID NO. 1 or a related sequence as described above in an assay as described above will allow for the analysis whether or not said target polypeptide has transmembrane pore formation capability or not, and if so, to what extent.

A transmembrane pore formation capability will be given in case that presence of the target polypeptide will result in increased calcium ion influx from outside the cell into the cytosol of said cell.

Therefore, the target polypeptide can be a transmembrane channel or a part of a transmembrane channel itself. Preferably, the target polypeptide is a transmembrane ion channel or part of a transmembrane ion channel itself. In case that the target polypeptide will be a part of a transmembrane ion channel, said part will usually be essential for the function of the transmembrane ion channel.

Alternatively, the target polypeptide can exhibit its transmembrane pore formation capability through interaction with one or more cellular receptors. In this case, the binding of the target polypeptide to the receptor will trigger the transmembrane pore formation. In a preferred embodiment, the target polypeptide exhibits its transmembrane pore formation capability through interaction with at least one insect receptor which is endogenous or heterologously expressed in the cell, preferably insect cell. Because of the expression of said insect receptor, said cell, preferably insect cell, becomes susceptible for the target polypeptide.

In a preferred embodiment, the target polypeptide is an insecticidal protein. More preferred, the target polypeptide is an insecticidal protein derived from the entomopathogenic bacterium Bacillus thuringiensis (Bt).

Bacillus thuringiensis (Bt) is a soil-borne bacterium that produces insecticidal crystal proteins known as delta endotoxins, or Cry proteins (Schnepf et al., Microbiol Mol Biol Rev. 1998 September; 62(3):775-806). Many B. thuringiensis serovars exist in nature that together account for a large number of diverse Cry proteins with various insecticidal properties. Cry proteins are oral intoxicants that function by acting on midgut cells of susceptible insects.

Particularly preferred, the target polypeptide is an insecticidal protein which comprises an amino acid sequence represented by SEQ ID NO. 4, SEQ ID NO. 5 or SEQ ID NO. 6, or comprises an amino acid sequence which has at least 80% sequence identity with one of the amino acid sequences represented by SEQ ID NO. 4, SEQ ID NO. 5 or SEQ ID NO. 6 over its entire length.

In another embodiment, the target polypeptide is an insecticidal protein which comprises an amino acid sequence which has at least 81%, 82%, 83%, 84%, 85%, 86%, 87%, 88%, 89%, 90%, 91%, 92%, 93%, 94%, 95%, 96%, 97%, 98%, 99% or 99.5% sequence identity with the amino acid sequence represented by SEQ ID NO. 4 over its entire length.

In another embodiment, the target polypeptide is an insecticidal protein which comprises an amino acid sequence which has at least 81%, 82%, 83%, 84%, 85%, 86%, 87%, 88%, 89%, 90%, 91%, 92%, 93%, 94%, 95%, 96%, 97%, 98%, 99% or 99.5% sequence identity with the amino acid sequence represented by SEQ ID NO. 5 over its entire length.

In another embodiment, the target polypeptide is an insecticidal protein which comprises an amino acid sequence which has at least 81%, 82%, 83%, 84%, 85%, 86%, 87%, 88%, 89%, 90%, 91%, 92%, 93%, 94%, 95%, 96%, 97%, 98%, 99% or 99.5% sequence identity with the amino acid sequence represented by SEQ ID NO. 6 over its entire length.

The amino acid sequences of SEQ ID NOs 4, 5 and 6 are the core sequences of the corresponding full-length proteins (protoxins) comprising the amino acid sequences of SEQ ID NOs 7, 8 and 9, respectively. It is these core sequences that are functionally active and therefore mediate the toxic activity. Preferably, said core sequences are obtained by trypsinization of the full-length proteins and follow-up purification (e.g. as described by Wang et al., Appl Environ Microbiol. 2019 Aug. 1; 85(16):e00579-19). Such trypsinization mimics the processes in the insect after ingestion of the protoxins: in the digestive tract of the insect, the protoxin dissolves in the alkaline environment and becomes susceptible for insect-specific proteases, e.g. trypsin, which cleave the protoxin and free its active toxic center, which in turn exhibits its toxic activity. Therefore, the trypsinization of the insecticidal protein, especially the trypsinization of an insecticidal protein comprising an amino acid sequence which is selected from SEQ ID NO 7, 8 and 9, or an amino acid sequence which has at least 80% sequence identity with one of said amino acid sequences over the entire length, leads to the activated form (tryptic core) of the insecticidal protein, i.e. the form that is capable of transmembrane pore formation or triggering transmembrane pore formation.

Another subject of the invention is a non-human host cell comprising a polypeptide as defined above and a target polypeptide as defined above. As described above, the effect of the addition of the target polypeptide to the cells, e.g. in a cellular assay, is the pore formation in the cellular membrane allowing calcium ions to enter the cytosol from the outside. Therefore, it is understood that with pore formation triggered by the target polypeptide, the non-human host cell is regarded to comprise said target polypeptide as well.

In one embodiment, the host cell is a native insect cell, i.e. the only manipulation of said insect cell is the expression of a polypeptide as defined above.

In another embodiment, the host cell is an insect cell which heterologously expresses at least one insect receptor gene, i.e. said at least one insect receptor gene is expressed in addition to the expression of a polypeptide as defined above.

The polypeptide to be used according to the invention is very suitable in cellular assays. Therefore, another subject of the invention is a cellular assay comprising
(i) a polypeptide as defined above and a target polypeptide as defined above, or
(ii) a host cell as defined above.

Further, another subject of the invention is a method for identifying transmembrane pore formation capability of a target polypeptide in a cellular assay, comprising the step of contacting said target polypeptide with a non-human cell which comprises a polypeptide as defined above.

According to the invention, the sequence identity values are determined using the BLASTX/ClustalW alignment tool.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. In another embodiment, the percent identity is calculated across the entirety of the reference sequence. The percent identity between two sequences can be determined using techniques similar to those described below, with or without allowing gaps. In calculating percent identity, typically exact matches are counted. A gap, i.e. a position in an alignment where a residue is present in one sequence but not in the other, is regarded as a position with non-identical residues.

The determination of percent identity between two sequences can be accomplished using a mathematical algorithm. A nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the BLASTN and BLASTX programs of Altschul et al. (1990) J. Mol. Biol. 215:403. BLAST nucleotide searches can be performed with the BLASTN program, score=100, wordlength=12, to obtain nucleotide sequences homologous to pesticidal-like nucleic acid molecules of the invention. BLAST protein searches can be performed with the BLASTX program, score=50, wordlength=3, to obtain amino acid sequences homologous to protein molecules according to the invention. To obtain gapped alignments for comparison purposes, Gapped BLAST (in BLAST 2.0) can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., BLASTX and BLASTN) can be used. Alignment may also be performed manually by inspection.

Another non-limiting example of a mathematical algorithm utilized for the comparison of sequences is the ClustalW algorithm (Higgins et al. (1994) Nucleic Acids Res. 22:4673-4680). ClustalW compares sequences and aligns the entirety of the amino acid or DNA sequence, and thus can provide data about the sequence conservation of the entire amino acid sequence. The ClustalW algorithm is used in several commercially available DNA/amino acid analysis software packages, such as the ALIGNX module of the Vector NTI Program Suite (Invitrogen Corporation, Carlsbad, CA). After alignment of amino acid sequences with ClustalW, the percent amino acid identity can be assessed. A non-limiting example of a software program useful for analysis of ClustalW alignments is GENEDOC™. GENEDOC™ (Karl Nicholas) allows assessment of amino acid (or DNA) similarity and identity between multiple proteins. Another non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG Wisconsin Genetics Software Package, Version 10 (available from Accelrys, Inc., 9685 Scranton Rd., San Diego, CA, USA). When utilizing the ALIGN program for comparing amino acid sequences, a PAM120 weight residue table, a gap length penalty of 12, and a gap penalty of 4 can be used.

Unless otherwise stated, Geneious 10, which uses the algorithm of Needleman and Wunsch (1970) J. Mol. Biol. 48(3):443-453, will be used to determine sequence identity or similarity using the following parameters: % identity for a nucleotide sequence using Gap open cost of 15 and Gap extend cost of 6.66, using the Clustal W2.1 cost matrix; % identity or % similarity for an amino acid sequence using Gap open cost of 20 and Gap extend cost of 0.1, using the BLOSUM cost matrix. Equivalent programs may also be used. By “equivalent program” is intended any sequence comparison program that, for any two sequences in question, generates an alignment having identical nucleotide residue matches and an identical percent sequence identity when compared to the corresponding alignment generated by Geneious 10.

### FIGURES

FIG. 1: Mode of action characterization of insecticidal proteins in a cell assay: Real time Cry-Protein activity measurement and pore formation by influx of calcium and genetically encoded calcium indicator of SEQ ID NO. 1 (GCaMP).

FIG. 2: Assay Workflow

FIG. 3: Functional characterization of SEQ ID NO. 1 (GCaMP) in recombinant Sf-9 cells. A: Fast calcium increase and increase of the fluorescence signal after addition of ionophore measured by genetically encoded Calcium indicator of SEQ ID NO.1 (GCaMP). B: No calcium increase after addition of Cry-Protein in Sf-9 cells expressing genetically encoded Calcium indicator of SEQ ID NO. 1 (GCaMP).

FIG. 4: Kinetic measurement of Ca²⁺ entry after addition of tryptic core insect toxins comprising SEQ ID NO. 4 (Cry1Ab) (A), SEQ ID NO. 5 (Cry1F) (B) and SEQ ID NO. 6 (Cry1A.105) (C) in Sf-9 cells expressing a receptor comprising an amino acid sequence of SEQ ID NO. 2 (ABCC2) by genetically encoded Calcium indicator of SEQ ID NO. 1 (GCaMP). Fast and concentration dependent Ca²⁺ influx and increase of the fluorescence signal after addition of activated insecticidal toxin in Sf-9 cells expressing a receptor with an amino acid sequence of SEQ ID NO. 2 (ABCC2).

FIG. 5: Dose response curves of a tryptic core insect toxin comprising SEQ ID NO. 6 (Cry1A.105)-protein activity on cells expressing a receptor with an amino acid sequence of SEQ ID NO. 3 (ABCC3) using GCaMP (SEQ ID NO. 1) read out.

FIG. 6: Kinetic measurement of Ca²⁺ entry and increase of the fluorescence signal after addition (5 μg/ml) of tryptic core insect toxin comprising SEQ ID NO. 5 (Cry1F) in Sf-9 cells expressing a receptor with an amino acid sequence of SEQ ID NO. 11 (ABCC2-B) or the respective mutated receptor with an amino acid sequence of SEQ ID NO. 10 (ABCC2 GY deletion; Boaventura et al., Insect Biochemistry and Molecular Biology 116, 2020) by genetically encoded Calcium indicator of SEQ ID NO. 1 (GCaMP). Fast Ca²⁺ influx after addition of activated insecticidal toxin in Sf-9 cells expressing a wild type receptor with an amino acid sequence of SEQ ID NO. 11 (ABCC2-B). No Ca²⁺ influx after addition of activated insecticidal toxin in Sf-9 cells expressing a mutated receptor with an amino acid sequence of SEQ ID NO. 10 (ABCC2 GY deletion).

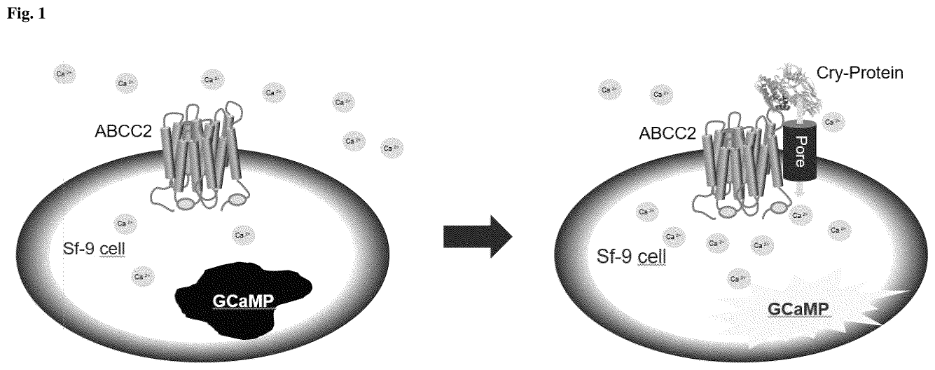

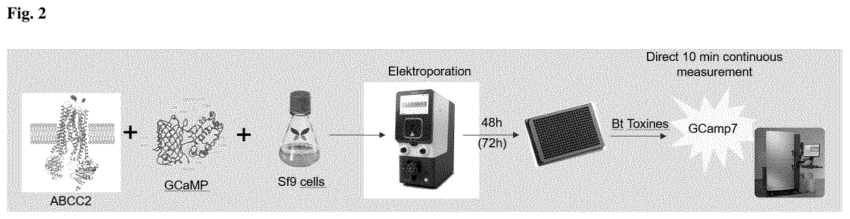

Without further elaboration, it is believed that one skilled in the art using the preceding description can utilize the present disclosure to its fullest extent. The following Example is, therefore, to be construed as merely illustrative, and not limiting of the disclosure in any way whatsoever.

### EXAMPLE

Sf-9 cells from Spodoptera frugiperda (Thermo Fisher Scientific) were transiently transfected by electroporation with plasmid DNA coding for putative insecticidal receptor proteins to express these recombinantly. However, the method of genetically encoded calcium ion (Ca²⁺) indicators can be applied also to insect gut primary cells expressing toxin receptors endogenously.

### Recombinant Insect Cell Assays

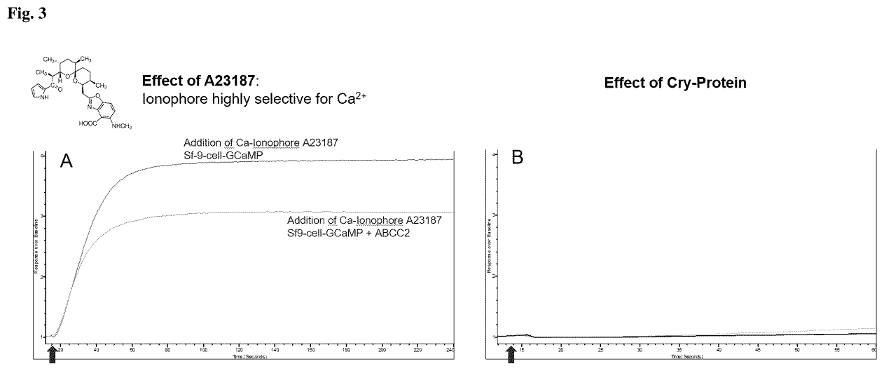

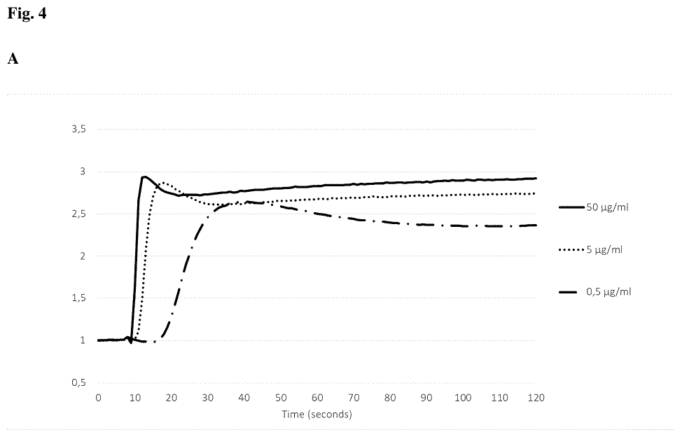

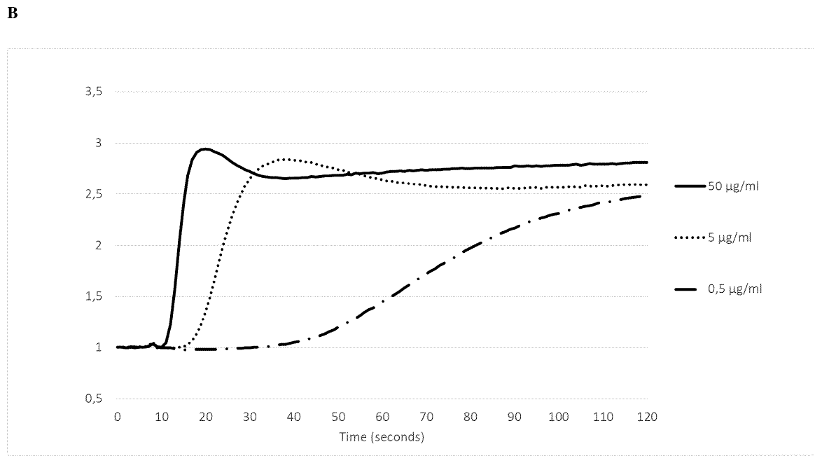

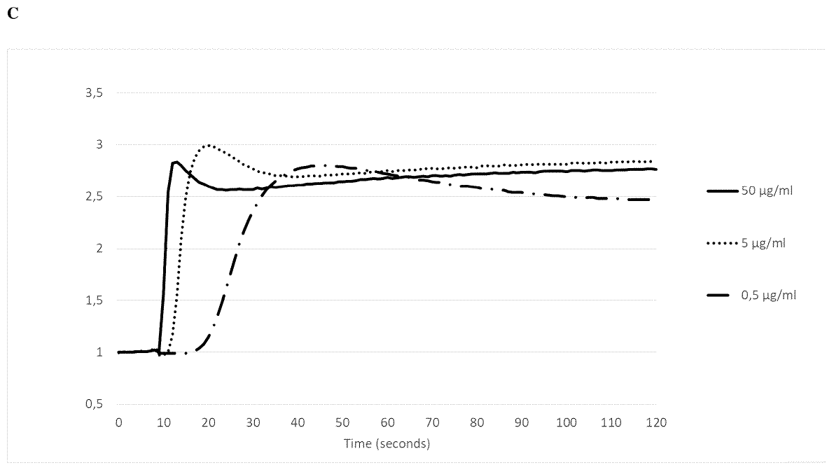

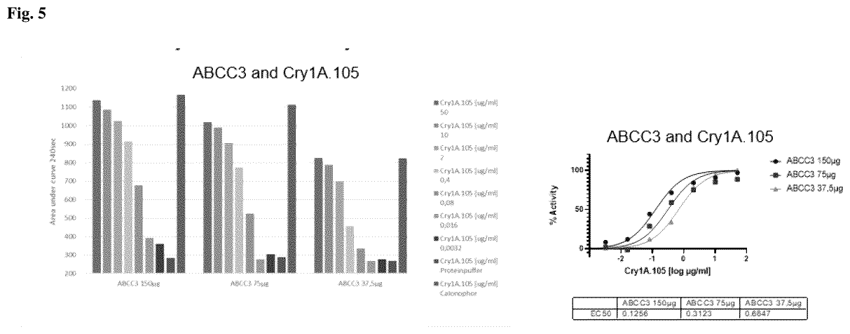

Sf-9 insect cells originally derived from ovarian cells of Spodoptera frugiperda were used to assess receptor function in genetically encoded calcium ion (Ca²⁺) indicator assays. As a control experiment, Sf-9 cells were only transfected with an expression-plasmid-DNA construct encoding SEQ ID NO. 1 demonstrating that these wild type cells do not respond to insecticidal proteins (FIG. 3B).

For the activity measurement experiment, the cells were transfected (MaxCyte) with two plasmid-DNA expression constructs, one encoding the polypeptide of SEQ ID NO. 1 and another one encoding a toxin receptor protein. The cells were distributed in 384 well plates including medium and kept in a humidified environment to prevent evaporation and incubated at 27° C. for 48 h. The medium was manually removed, then plates were loaded with standard Tyrode Buffer. Plates were analyzed at FLIPR-TETRA (EMCCD-Camera) using a λexc 470-495 nM/λem 515-575 nM filter. The wells were injected with the indicated protein toxins (tryptic core) and fluorescence was measured for 5 min after injection.

### Data Analysis

All the FLIPR-TETRA measurements were analyzed with Screenworks© software (Molecular Devices, Version 4.0) and data were exported as area under curve Statistics calculated after compound injection.

Absolute Response (RFU) was obtained applying “Response over baseline”, while baseline start with the first and end with the second timepoint of measurement before injection.

### Results

Sf-9 cells expressing SEQ ID NO. 1 only showed no fluorescence signal when insect toxin (SEQ ID NO. 5) was added (FIG. 3B). However, addition of a calcium ionophore like A23187 on these cells showed a significant fluorescence signal due to the intracellular calcium increase and binding of calcium to the polypeptide of SEQ ID NO. 1 (FIG. 3A).

Sf-9 cells expressing the polypeptide of SEQ ID NO. 1 and an insect toxin receptor (SEQ ID NO. 2) showed a fast and concentration dependent increase of the fluorescence signal when purified, tryptic core insect toxin (comprising SEQ ID NO. 4, SEQ ID NO. 5 or SEQ ID NO. 6 derived from full length proteins (protoxins) of SEQ ID NO. 7, SEQ ID NO. 8 and SEQ ID NO. 9, respectively, after trypsinization) was added to the cells indicating toxin-induced membrane permeabilization and calcium increase. Another insect toxin receptor (SEQ ID NO. 3) responded to SEQ ID NO. 6.

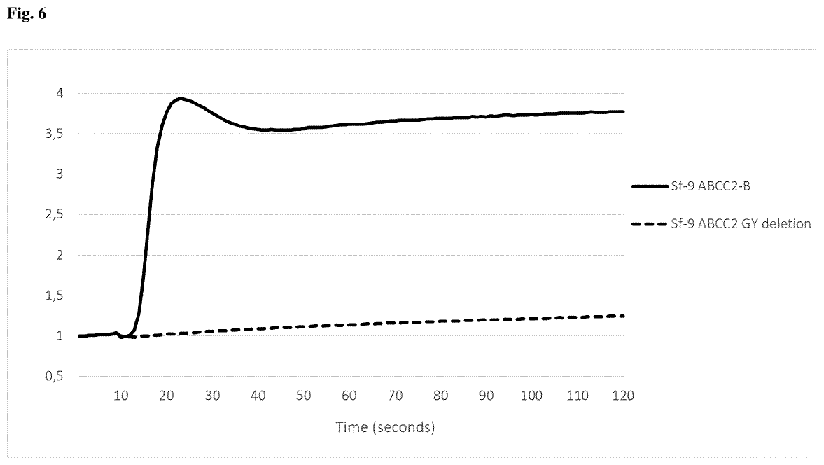

Further, mutated insect toxin receptors (SEQ ID NO. 10) which result in field resistance do not respond to insect toxins comprising SEQ ID NO. 5. Therefore, this experimental approach enables also for resistance research activities to validate target site mutations in expressed receptor proteins (FIG. 6).

1. A method for identifying a transmembrane pore formation capability of a target polypeptide in a cellular assay, the method comprising use of a polypeptide, wherein the polypeptide comprises one of:
(i) an amino acid sequence represented by SEQ ID NO. 1, or
(ii) an amino acid sequence which has at least 80% sequence identity with the amino acid sequence of (i) over its entire length as determined using the BLASTX/ClustalW alignment tool, wherein said amino acid sequence has calcium ion (Ca²⁺) indicator activity.

2. The method according to claim 1, wherein the polypeptide provides a fluorescent signal upon calcium ion (Ca²⁺) binding.

3. The method according to claim 1, wherein cells in the cellular assay are native insect cells.

4. The method according to claim 1, wherein cells in the cellular assay are cells which heterologously express at least one insect receptor gene.

5. The method according to claim 3, wherein the cells in the cellular assay comprise an insect receptor polypeptide comprising one of:
(i) an amino acid sequence represented by SEQ ID NO. 2 or SEQ ID NO. 3 or SEQ ID NO. 10 or SEQ ID NO. 11, or
(ii) comprises an amino acid sequence which has at least 80% sequence identity with one of the amino acid sequences of (i) over its entire length as determined using the BLASTX/ClustalW alignment tool.

6. The method according to claim 1, wherein the target polypeptide is a transmembrane channel or a part of a transmembrane channel.

7. The method according to claim 1, wherein the target polypeptide exhibits transmembrane pore formation capability through interaction with at least one cellular receptor.

8. The method according to claim 7, wherein the at least one cellular receptor:
(i) comprises an amino acid sequence represented by SEQ ID NO. 2 or SEQ ID NO. 3 or SEQ ID NO. 10 or SEQ ID NO. 11, or
(ii) comprises an amino acid sequence which has at least 80% sequence identity with one of the amino acid sequences of (i) over its entire length as determined using the BLASTX/ClustalW alignment tool.

9. The method according to claim 1, wherein the target polypeptide is an insecticidal protein.

10. The method according to claim 9, wherein the target polypeptide is insecticidal protein derived from Bacillus thuringiensis.

11. The method according to claim 1, wherein the target polypeptide is an insecticidal protein which comprises an amino acid sequence represented by SEQ ID NO. 4, SEQ ID NO. 5 or SEQ ID NO. 6, or comprises an amino acid sequence which has at least 80% sequence identity with one of the amino acid sequences represented by SEQ ID NO. 4, SEQ ID NO. 5 or SEQ ID NO. 6 over its entire length.

12. A non-human host cell comprising a polypeptide and a target polypeptide each comprising one of:
(i) an amino acid sequence represented by SEQ ID NO. 1, or
(ii) an amino acid sequence which has at least 80% sequence identity with the amino acid sequence of (i) over its entire length as determined using the BLASTX/ClustalW alignment tool, wherein said amino acid sequence has calcium ion (Ca²⁺) indicator activity.

13. The host cell according to claim 12, wherein said host cell is a native insect cell.

14. The host cell according to claim 12, wherein said host cell is a cell which heterologously expresses at least one insect receptor gene.

15. A cellular assay comprising one of:
(i) a polypeptide and a target polypeptide each comprising one of:
(iia) an amino acid sequence represented by SEQ ID NO. 1, or
(iib) an amino acid sequence which has at least 80% sequence identity with the amino acid sequence of (i) over its entire length as determined using the BLASTX/ClustalW alignment tool, wherein said amino acid sequence has calcium ion (Ca²⁺) indicator activity, or
(ii) a host cell as defined in claim 12.

16. A method for identifying transmembrane pore formation capability of a target polypeptide in a cellular assay, the method comprising contacting said target polypeptide with a non-human cell which comprises a polypeptide as defined in claim 1.


SEQUENCE LISTING (11 sequences)

SEQ ID NO: 1 - protein, 442 aa, synthetic construct
MGSASMTGGQQMGRDLYDDDDKDLATMVDSSRRKWNKTGHAVRAIGRLSSLENVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNTRDQLTEEQIAEFKEAFSLFDKDGDGTITTKELGTVMRSLGQNPTEAELQDMINEVDADGDGTIDFPEFLTMMARKMKDTDSEEEIREAFRVFDKDGNGYISAAELRHVMTNLGEKLTDEEVDEMIREADIDGDGQVNYEEFVQMMTAK

SEQ ID NO: 2 - protein, 1357 aa, Spodoptera frugiperda
MMDKSNKNTAANGNGGQRAGEPKERVRKKPNILSRIFVWWIFPVLITGNKRDVEEDDLIVPSKKFNSERQGEYFERYWFEEVAIAEREDRDPSLWKAMRRAYWLQYMPGAIFVLLISGLRTAQPLLFSQLLSYWSVDSEMSQQDAGLYALAMLGINFITMMCTHHNNLFVMRFSMKVKIAASSLLFRKLLRMSQVSVGDVAGGKLVNLLSNDVARFDYAFMFLHYLWVVPLQVGVVLYFVYDAAGWAPYVGLFGVIILIMPLQAGLTKLTGVVRRMTAKRTDKRIKLMSEIINGIQVIKMYAWEKPFQLVVKAARAYEMSALRKSIFIRSMFLGFMLFTERSVMFLTVLTLALTGNMISATLIYPIQQYFGIITMNVTLILPMAFASFSEMLISLERIQGFLLLDERSDIQITPKVVNGAGSKLFNNSKKEGGLETGIVLPTKYSPTEANIARPMQDEPNMADYPVQLNKVNATWADLNDNKEMTLKNISLRVRKNKLCAVIGPVGSGKTSLLQLLLRELPVTSGNLSISGTVSYASQEPWLFPATVRENILFGLEYNVAKYKEVCKVCSLLPDFKQFPYGDLSLVGERGVSLSGGQRARINLARAVYREADIYLLDDPLSAVDANVGRQLFDGCIKGYLSGKTCILVTHQIHYLKAADFIVVLNEGSVENMGSYDELMKTGTEFSMLLSDQASEGSDTDKKERPAMMRGISKMSVKSDDEEGEEKVQVLEAEERQSGSLKWDVLGRYMKSVNSWCMVVMAFLVLVITQGAATTTDYWLSFWTNQVDGYIQTLPEGESPNPELNTQVGLLTTGQYLIVHGSVVLAIIILTQVRILSFVVMTMRASENLHNTIYEKLIVAVMRFFDTNPSGRVLNRFSKDMGAMDELLPRSMLETVQMYLSLASVLVLNAIALPWTLIPTTVLMFIFVFLLKWYINAAQAVKRLEGTTKSPVFGMINSTISGLSTIRSSNSQDRLLNSFDDAQNLHTSAFYTFLGGSTAFGLYLDTLCLIYLGIIMSIFILGDFGELIPVGSVGLAVSQSMVLTMMLQMAAKFTADFLGQMTAVERVLEYTKLPTEENMETGPTTPPKGWPSAGEVTFSNVYLKYSPDDPPVLKDLNFAIKSGWKVGVVGRTGAGKSSLISALFRLSDITGSIKIDGLDTQGIAKKLLRSKISIIPQEPVLFSASLRYNLDPFDNYNDDDIWRALEQVELKESIPALDYKVSEGGTNFSMGQRQLVCLARAILRSNKILIMDEATANVDPQTDALIQKTIRKQFATCTVLTIAHRLNTIMDSDRVLVMDQGVAAEFDHPYILLSNPNSKFSSMVKETGDNMSRILFEVAKTKYESDSKTADYKDDDDK

SEQ ID NO: 3 - protein, 1367 aa, Spodoptera frugiperda
MDTKKGSKNDSKSKPPPPKPKSVPNVFKRTLFCWMLPIFYFGNRRDLEESDLPPPKNMYQSKMLGDKLERSWLKEEHEAKLAGRKPKFTKVLFKTFIWSYIPGGLMQATSIGLRTASPLLFSQLLRYWAADSPVDRETAMYYAISMILANWASSFMNHHGVLFCQQFGMKLRCAVGSLMFRKIMRMSNGSLGDTAAGKVVNILSNDLQRFDLSMVFLHYVWIIPLQIAAVIYLGYLQAGTAAFIGFAALIIIALPFQGGLGQYLGKIRLRTAEKTDNRIKIMSEVINGIQVIKMYAWEIPFQKVVGQKRAEELKEVKIATILRTVFLGFMMFTERAALFFTVLTYVLLGNVMSANVFYPLQQFMSAAQVNITLILPMVLSFTAELLVSLGRVEKFLLLEDRPDLKGHPEETSSNLFRNMSADGIPETNGSIRPLSYHRKSEASPIEPGSEIQLKVPKFTRSVSYQEDSALVLHDVSASWTGDPNMMALKNISMRLRKGKLCAIIGAVGSGKSSVLQLLLKELPSATGTISIYGKMSYACQEAWLFPSTVRENILFGLPFEPGKYKKVCRVCALEKDFKQFPYGDQTLVGERGVSLSGGQRARINLARAVYREADIYLLDDPLSAVDANVGRQLFEGCINGYLRGRTRILVTHQIHFLKAADYIVVLNEGRIENMGTFDELVSSGKEFSMMLAQLQEGKEKDTESVGSRSIEEKEKPTLKTMISVNEGEEVQEFEAQKMKEEERQSGNLRWEVISAYFRSGGHVCFILFALLVVVLATTCAASVDYWVSYWTNQMAAYEESLGGESIEPGLDVQAGRFTVGQYLTIHGCLVAALVLMVNLRVFPFAYLCVSASAKLHNQMFSTMLRGVMRFFDTSSSGRILNRFTKDMGSLDEILPRTLLDVLQIYGTLTAILVLNAIALYWTLVPSAVLLVLFGFMVRIYLKAAQGIKRLEGTTKSPMFGTVTSSLSGISTIRASNAQERLIEQFDINQDLHTTSWNSYLNGGTTFGFYLDTMCLVYMTTIIFVFLFIDFGDAIPVGSVGLAVTQSNTLTLMLQHGARMLVEFLAQLTSVERILEYTRIDTEPDLFQGKVEMPPNWPYQGRIEFQNVSLRYAPNEQPVLKNLNIVIESGNKIGIVGRTGAGKSSLISALFRFAYLDGLISIDGLDTSLISRQGLRSKISIIPQEPILFSATIRYNLDPFDIYSDDDLWRALEQVDLKSAVPSLDFKVTEGGSNFSVGQRQLMCLARAVLRSNQILIMDEATANVDPQTDNFIQETIRRQFVSCTVLTIAHRLNTIMDSDKVLVMSSGQVAEYDHPYVLLSDPNSHFSAMVRETGEKNSANLFQVAKDAYFQSNLKENARDYKDDDDK

SEQ ID NO: 4 - protein, 591 aa, Bacillus thuringiensis
IETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLER

SEQ ID NO: 5 - protein, 585 aa, Bacillus thuringiensis
STGRLPLDISLSLTRFLLSEFVPGVGVAFGLFDLIWGFITPSDWSLFLLQIEQLIEQRIETLERNRAITTLRGLADSYEIYIEALREWEANPNNAQLREDVRIRFANTDDALITAINNFTLTSFEIPLLSVYVQAANLHLSLLRDAVSFGQGWGLDIATVNNHYNRLINLIHRYTKHCLDTYNQGLENLRGTNTRQWARFNQFRRDLTLTVLDIVALFPNYDVRTYPIQTSSQLTREIYTSSVIEDSPVSANIPNGFNRAEFGVRPPHLMDFMNSLFVTAETVRSQTVWGGHLVSSRNTAGNRINFPSYGVFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEISGSDSWRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLER

SEQ ID NO: 6 - protein, 591 aa, Bacillus thuringiensis
IETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATLEAEYNLER

SEQ ID NO: 7 - protein, 1155 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSDEFCLDEKKELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAPSPIGKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDRLQADTNIAMIHAADKRVHSIREAYLPELSVIPGVNAAIFEELEGRIFTAFSLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTATQEEYEGTYTSRNRGYDGAYESNSSVPADYASAYEEKAYTDGRRDNPCESNRGYGDYTPLPAGYVTKELEYFPETDKVWIEIGETEGTFIVDSVELLLMEE

SEQ ID NO: 8 - protein, 1171 aa, Bacillus thuringiensis
MAENNIQNQCVPYNCLNNPEVEILNEERSTGRLPLDISLSLTRFLLSEFVPGVGVAFGLFDLIWGFITPSDWSLFLLQIEQLIEQRIETLERNRAITTLRGLADSYEIYIEALREWEANPNNAQLREDVRIRFANTDDALITAINNFTLTSFEIPLLSVYVQAANLHLSLLRDAVSFGQGWGLDIATVNNHYNRLINLIHRYTKHCLDTYNQGLENLRGTNTRQWARFNQFRRDLTLTVLDIVALFPNYDVRTYPIQTSSQLTREIYTSSVIEDSPVSANIPNGFNRAEFGVRPPHLMDFMNSLFVTAETVRSQTVWGGHLVSSRNTAGNRINFPSYGVFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEISGSDSWRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQKAVNALFTSINQIGIKTDVTDYHIDQVSNLVDCLSDEFCLDEKRELSEKVKHAKRLSDERNLLQDPNFKGINRQLDRGWRGSTDITIQRGDDVFKENYVTLPGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCEFNRGYRDYTPLPVGYVTKELEYFPETDKVWIEIGETEGTFIVDSVELLLMEE

SEQ ID NO: 9 - protein, 1177 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATLEAEYNLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYSIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCEFNRGYRDYTPLPVGYVTKELEYFPETDKVWIEIGETEGTFIVDSVELLLMEE

SEQ ID NO: 10 - protein, 1369 aa, Spodoptera frugiperda
MMDKSNKNTAANGNGGQRAGEPKERVRKKPNILSRIFVWWIFPVLITGNKRDVEEDDLIVPSKKFNSERQGEYFERYWFEEVAIAEREDRDPSLWKAMRRAYWLQYMPGAIFVLLISGLRTAQPLLFSQLLSYWSVDSEMSQQDAGLYALAMLGINFITMMCTHHNNLFVMRFSMKVKIAASSLLFRKLLRMSQVSVGDVAGGKLVNLLSNDVARFDYAFMFLHYLWVVPLQVGVVLYFVYDAAGWAPYVGLFGVIILIMPLQAGLTKLTGVVRRMTAKRTDKRIKLMSEIINGIQVIKMYAWEKPFQLVVKAARAYEMSALRKSIFIRSMFLGFMLFTERSVMFLTVLTLALTGNMISATLIYPIQQYFGIITMNVTLILPMAFASFSEMLISLERIQGFLLLDERSDIQITPKVVNGAGSKLFNNSKKEGGLETGIVLPTKYSPTEANMARPMQDEPNMADYPVQLNKVNATWADLNDNKEMTLKNISLRVRKNKLCAVIGPVGSGKTSLLQLLLRELPVTSGNLSISGTVSYASQEPWLFPATVRENILFGLEYNVAKYKEVCKVCSLLPDFKQFPYGDLSLVGERGVSLSGGQRARINLARAVYREADIYLLDDPLSAVDANVGRQLFDGCIKGYLSGKTCILVTHQIHYLKAADFIVVLNEGSVENMGSYDELMKTGTEFSMLLSDQASEGSDTDKKERPAMMRGISKMSVKSDDEEGEEKVQVLEAEERQSGSLKWDVLGRYMKSVNSWCMVVMAFLVLVITQGAATTTDYWLSFWTNQVDIQTLPEGESPNPELNTQVGLLTTGQYLIVHGSVVLAIIILTQVRILSFVVMTMRASENLHNTIYEKLIVAVMRFFDTNPSGRVLNRFSKDMGAMDELLPRSMLETVQMYLSLASVLVLNAIALPWTLIPTTVLMFIFVFLLKWYINAAQAVKRLEGTTKSPVFGMINSTISGLSTIRSSNSQDRLLNSFDDAQNLHTSAFYTFLGGSTAFGLYLDTLCLIYLGIIMSIFILGDFGELIPVGSVGLAVSQSMVLTMMLQMAAKFTADFLGQMTAVERVLEYTKLPTEENMETGPTTPPKGWPSAGEVTFSNVYLKYSPDDPPVLKDLNFAIKSGWKVGVVGRTGAGKSSLISALFRLSDITGSIKIDGLDTQGIAKKLLRSKISIIPQEPVLFSASLRYNLDPFDNYNDEDIWRALEQVELKESIPALDYKVSEGGTNFSMGQRQLVCLARAILRSNKILIMDEATANVDPQTDALIQKTIRKQFATCTVLTIAHRLNTIMDSDRVLVMDQGVAAEFDHPYILLSNPNSKFSSMVKETGDNMSRILFEVAKTKYESDSKTADYKDHDGDYKDHDIDYKDDDDK

SEQ ID NO: 11 - protein, 1371 aa, Spodoptera frugiperda
MMDKSNKNTAANGNGGQRAGEPKERVRKKPNILSRIFVWWIFPVLITGNKRDVEEDDLIVPSKKFNSERQGEYFERYWFEEVAIAEREDRDPSLWKAMRRAYWLQYMPGAIFVLLISGLRTAQPLLFSQLLSYWSVDSEMSQQDAGLYALAMLGINFITMMCTHHNNLFVMRFSMKVKIAASSLLFRKLLRMSQVSVGDVAGGKLVNLLSNDVARFDYAFMFLHYLWVVPLQVGVVLYFVYDAAGWAPYVGLFGVIILIMPLQAGLTKLTGVVRRMTAKRTDKRIKLMSEIINGIQVIKMYAWEKPFQLVVKAARAYEMSALRKSIFIRSMFLGFMLFTERSVMFLTVLTLALTGNMISATLIYPIQQYFGIITMNVTLILPMAFASFSEMLISLERIQGFLLLDERSDIQITPKVVNGAGSKLFNNSKKEGGLETGIVLPTKYSPTEANMARPMQDEPNMADYPVQLNKVNATWADLNDNKEMTLKNISLRVRKNKLCAVIGPVGSGKTSLLQLLLRELPVTSGNLSISGTVSYASQEPWLFPATVRENILFGLEYNVAKYKEVCKVCSLLPDFKQFPYGDLSLVGERGVSLSGGQRARINLARAVYREADIYLLDDPLSAVDANVGRQLFDGCIKGYLSGKTCILVTHQIHYLKAADFIVVLNEGSVENMGSYDELMKTGTEFSMLLSDQASEGSDTDKKERPAMMRGISKMSVKSDDEEGEEKVQVLEAEERQSGSLKWDVLGRYMKSVNSWCMVVMAFLVLVITQGAATTTDYWLSFWTNQVDGYIQTLPEGESPNPELNTQVGLLTTGQYLIVHGSVVLAIIILTQVRILSFVVMTMRASENLHNTIYEKLIVAVMRFFDTNPSGRVLNRFSKDMGAMDELLPRSMLETVQMYLSLASVLVLNAIALPWTLIPTTVLMFIFVFLLKWYINAAQAVKRLEGTTKSPVFGMINSTISGLSTIRSSNSQDRLLNSFDDAQNLHTSAFYTFLGGSTAFGLYLDTLCLIYLGIIMSIFILGDFGELIPVGSVGLAVSQSMVLTMMLQMAAKFTADFLGQMTAVERVLEYTKLPTEENMETGPTTPPKGWPSAGEVTFSNVYLKYSPDDPPVLKDLNFAIKSGWKVGVVGRTGAGKSSLISALFRLSDITGSIKIDGLDTQGIAKKLLRSKISIIPQEPVLFSASLRYNLDPFDNYNDEDIWRALEQVELKESIPALDYKVSEGGTNFSMGQRQLVCLARAILRSNKILIMDEATANVDPQTDALIQKTIRKQFATCTVLTIAHRLNTIMDSDRVLVMDQGVAAEFDHPYILLSNPNSKFSSMVKETGDNMSRILFEVAKTKYESDSKTADYKDHDGDYKDHDIDYKDDDDK